(19) United States
(12) Patent Application Publication
(10) Pub. No.: US 2024/0047052 A1
(43) Pub. Date: Feb. 8, 2024

(54) SYSTEM AND METHOD FOR FORECASTING STAFFING LEVELS IN AN INSTITUTION

(71) Applicant: SCP Health, Lafayette, LA (US)

(72) Inventors: Nicholas Jordan Ruiz, Lafayette, LA (US); Jeremy Charles Martin, Baton Rouge, LA (US); Robert Barry Reilly, Marietta, GA (US)

(21) Appl. No.: 18/364,184

(22) Filed: Aug. 2, 2023

Related U.S. Application Data
(60) Provisional application No. 63/394,517, filed on Aug. 2, 2022.

(51) Int. Cl. G16H 40/20

(52) U.S. Cl. CPC G16H 40/20

(57) ABSTRACT

A system and method that utilizes machine learning, volume prediction, input staffing and acuity data, and simulation data to align staffing coverage with forecasted coverage needs of an institution, such as a healthcare facility. The system can better match staffing coverage with staffing needs and patient demand resulting in cost savings and reducing the risk of overworking existing staff.

### RELATED APPLICATION

The present application claims priority to U.S. provisional patent application Ser. No. 63/394,517, filed on Aug. 2, 2022, and entitled SYSTEM AND METHOD FOR FORECASTING STAFFING LEVELS IN AN INSTITUTION, the contents of which are herein incorporated by reference.

### BACKGROUND OF THE INVENTION

The present application relates to forecasting methods, and more specifically relates to systems and methods for forecasting staffing levels in a healthcare facility.

In institutions, such as companies, employees oftentimes are the greatest asset. As such, ensuring that the company is appropriately staffed helps ensure adequate coverage to meet the needs of the enterprise or business, while concomitantly ensuring that the enterprise is not overstaffed or understaffed. If the enterprise is overstaffed, then the costs to the enterprise suffer and the employees can suffer from boredom. If the enterprise is understaffed to meet the overall business needs, then the enterprise risks overworking their employees, which can result in employee burn-out and high levels of employee turnover. As such, it is important to create and adopt a reasonable staffing or coverage plan. A staffing or coverage plan is a plan that results from a strategic planning process by which an enterprise assesses and identifies the personnel needs of the enterprise. In other words, a good staffing plan helps the enterprise understand the number and types of employees the enterprise needs to accomplish its business and other types of goals. The traditional staffing plan typically determines the types of work that need to be performed, how many people are needed to handle the work, and the relative skill levels and backgrounds of the people required to do the work. The staffing plan can encompass the entire enterprise or apply to smaller teams or departments and even individual projects within the enterprise. A well-constructed staffing plan can help reduce labor costs and maximize productivity, eliminate skill gaps, increase employee engagement, increase employee retention, reduce turnover, and improve the customer experience.

The conventional staffing plans are time consuming and difficult to create. The conventional staffing plans are typically created manually by people using historical staffing levels and data as a guide, as well as considering one or more additional factors that can affect overall staffing levels, such as any related business goals or objectives, turnover rates and projections, industry labor costs, and the like. As such, because of the laborious process and the sophistication of the overall process, the manually created staffing plans tend to be inadequate and cannot fully assess all relevant factors that can impact the staffing plan.

### SUMMARY OF THE INVENTION

The present invention is directed to a dynamic system and method that utilizes machine learning, volume prediction, input staffing and acuity data, and simulation data to align staffing coverage with forecasted coverage needs of an institution. As such, the system of the present invention can better match staffing coverage with staffing needs and demand resulting in cost savings and reducing the risk of overworking existing staff.

The present invention is directed to a computer implemented coverage determination system for determining a coverage plan for a healthcare facility. The system can include a processor and a non-transitory memory having instructions configuring the processor to: train a forecasting model with historical patient volume data as an input and correlating the historical patient volume data with the healthcare facility, where the trained forecasting model generates patient volume forecast data in response to the patient volume data and the forecasting model includes a plurality of hyperparameters and the patient volume data covers a patient volume over a first selected period of time; tuning one or more of the plurality of hyperparameters by applying thereto a tuning technique, wherein the tuning unit generates in response tuned patient volume forecast data; generating with a simulation engine simulation data in response to the tuned patient volume forecast data and acuity level data where the simulation data includes the patient volume over a second period of time, wherein the second period of time is shorter than the first period of time; and generating a coverage plan in response to and based on the simulation data, coverage data received from a data storage unit, and coverage rules data from a coverage rules unit.

The simulation engine simulates the coverage plan based on the tuned patient volume forecast data, the acuity level data, and lay-out data of the healthcare facility. The simulation data includes a visual representation of a coverage requirement of one or more portions of the healthcare facility. The coverage data can include any combination of default schedule data, contract staffing requirement data, relative value unit capacity data, service provider type data, cost by provider type data, additional acuity level data, predefined staffing requirements based on the acuity level, contract staffing rules data, and patient treatment related data. The coverage rules data can include any combination of a length of a coverage shift, one or more time period limitations on the shift, and a predetermined number of shifts per healthcare facility. As such, the coverage plan matches staff coverage needs in one or more selected time increments for a selected period of time with or for the healthcare facility. The coverage plan further includes one or more coverage recommendations based on coverage needs and costs per shift for the healthcare facility. The system can also include a user interface generator for generating in response to the coverage plan one or more user interfaces for displaying the coverage plan.

The present invention an also be directed to coverage determination system for determining a coverage plan for a healthcare facility comprising a forecasting unit for applying a forecasting model to patient volume data received from a data storage unit and then generating in response patient volume forecast data, where the forecasting model includes a plurality of hyperparameters and the patient volume data covers a patient volume over a first selected period of time; a tuning unit for tuning one or more of the plurality of hyperparameters by applying thereto a tuning technique, wherein the tuning unit generates in response tuned patient volume forecast data; a simulation engine for receiving the tuned patient volume forecast data and acuity level data and then generating in response thereto simulation data, where the simulation data includes the patient volume over a second period of time and where the second period of time is shorter than the first period of time; and a coverage determination unit for receiving the simulation data, coverage data from the data storage unit, and coverage rules data from a coverage rules unit, and then generating in response thereto the coverage plan. The learning forecasting model can include Facebook Prophet or ARIMA, and the tuning technique can include a machine learning R model based optimization (mlrMBO) technique.

The simulation engine simulates the coverage plan based on the tuned patient volume forecast data, the acuity level data, and lay-out data of the healthcare facility. The simulation data includes a visual representation of a coverage requirement of one or more portions of the healthcare facility, and the first period of time is 60 days and the second period of time is 30 minutes. The coverage data can include one or more of default schedule data, contract staffing requirement data, relative value unit capacity data, service provider types data, cost by provider type data, additional acuity level data, predefined staffing requirements based on the acuity level, contract staffing rules data, and patient treatment related data. The coverage rules data can include any combination of a length of a coverage shift, time period limitations on the shift, and predetermined number of shifts per healthcare facility. The coverage plan matches staff coverage needs in one or more selected time increments for a selected period of time, and can include one or more coverage recommendations based on coverage needs and costs per shift for the healthcare facility.

The system can further include a user interface generator for generating in response to the overage plan one or more user interfaces for displaying the coverage plan. The user interface generator generates a first user interface having a first window configured for displaying a selected calendar view in a calendar format, where each day of the calendar view can display therein selected healthcare related data including a number of staff scheduled to work each day as well as the expected patient volume for each day. The first window can include an optimization soft button that when actuated optimizes the healthcare related data displayed in the calendar view. Further, when a selected day in the calendar view is selected by a user, the user interface generator generates a second interface having a second window having a plurality of vertically stacked pane elements for displaying various types of information. The plurality of vertically stacked pane elements can include an uppermost pane element that displays information associated with the coverage plan generated by the coverage determination unit, a second intermediate pane element for displaying selected staff coverage information associated with the coverage plan and RVU information, and a third lowermost pane element for displaying staffing information associated with the coverage plan, where the second pane element is between the first and third pane elements. The user interface generator can also generate a third user interface having a third window for displaying a forecast accuracy dashboard associating an accuracy value associated the patient volume or the staff coverage of the coverage plan for one or more days.

The present invention is also directed to a computer implemented method for determining a coverage plan for a healthcare facility, where the method includes: applying, using an electronic device, a forecasting model to patient volume data received from a data storage unit and then generating in response patient volume forecast data, where the forecasting model includes a plurality of hyperparameters and the patient volume data covers a patient volume over a first selected period of time; tuning, using the electronic device, one or more of the plurality of hyperparameters by applying thereto a tuning technique, wherein the tuning unit generates in response tuned patient volume forecast data; generating, using the electronic device and with a simulation engine, simulation data in response to the tuned patient volume forecast data and acuity level data, wherein the simulation data includes the patient volume over a second period of time, wherein the second period of time is shorter than the first period of time; and generating, using the electronic device, a coverage plan in response to and based on the simulation data, coverage data received from a data storage unit, and coverage rules data from a coverage rules unit. The method can also include training the forecasting model with patient volume data as an input and correlating the patient volume data with one or more selected parameters associated with the healthcare facility.

The method can also include simulating, with the simulation engine, the coverage plan based on the tuned patient volume forecast data, the acuity level data, and lay-out data of the healthcare facility. The simulation data includes a visual representation of a coverage requirement of one or more portions of the healthcare facility. The coverage data comprises any combination of default schedule data, contract staffing requirement data, relative value unit capacity data, service provider type data, cost by provider type data, additional acuity level data, predefined staffing requirements based on the acuity level, contract staffing rules data, and patient treatment related data. The coverage rules data comprises any combination of a length of a coverage shift, one or more time period limitations on the shift, and a predetermined number of shifts per healthcare facility. The coverage plan matches staff coverage needs in one or more selected time increments for a selected period of time with the healthcare facility, and can include one or more coverage recommendations based on coverage needs and costs per shift for the healthcare facility.

The present invention can also be directed to a non-transitory, computer readable medium comprising computer program instructions tangibly stored on the computer readable medium, wherein the computer program instructions are executable by at least one computer processor to perform a method, the method comprising: applying a forecasting model to patient volume data received from a data storage unit and then generating in response patient volume forecast data, where the forecasting model includes a plurality of hyperparameters and the patient volume data covers a patient volume over a first selected period of time; tuning one or more of the plurality of hyperparameters by applying thereto a tuning technique, wherein the tuning unit generates in response tuned patient volume forecast data; generating with a simulation engine simulation data in response to the tuned patient volume forecast data and acuity level data, where the simulation data includes the patient volume over a second period of time, wherein the second period of time is shorter than the first period of time; and generating a coverage plan in response to and based on the simulation data, coverage data received from a data storage unit, and coverage rules data from a coverage rules unit. The medium can also include training the forecasting model with historical patient volume data as an input and correlating the historical patient volume data with the healthcare facility.

### BRIEF DESCRIPTION OF THE DRAWINGS

These and other features and advantages of the present invention will be more fully understood by reference to the following detailed description in conjunction with the attached drawings, in which like reference numerals refer to like elements throughout the different views. The drawings illustrate principals of the invention and, although not to scale, show relative dimensions.

FIG. 1 is a schematic block diagram illustrating the coverage determination system according to the teachings of the present invention.

FIG. 2 is a schematic flow chart diagram illustrating the process employed by the coverage determination system of the present invention.

FIG. 3 is a representation of a user interface of a default coverage plan generated by the coverage determination system of the present invention.

FIG. 4 is a representation of a user interface of an optimized coverage plan generated by the coverage determination system of the present invention.

FIG. 5 is a representation of a user interface of a drill down view showing additional information associated with a selected day default coverage plan generated by the coverage determination system of the present invention.

FIG. 6 is a representation of a user interface of a run history generated by the coverage determination system of the present invention.

FIG. 7 is a representation of a user interface showing a forecast accuracy dashboard generated by the coverage determination system of the present invention.

FIG. 8 is a representation of a user interface showing a look back view of a coverage plan generated by the coverage determination system of the present invention.

FIG. 9 is a representation of a user interface of a historical fit of a prior coverage plan generated by the coverage determination system of the present invention.

FIG. 10 is a schematic diagram of an electronic or computing device and/or associated system suitable for implementing the coverage determination system of the present invention.

### DETAILED DESCRIPTION

The present invention is directed to a system and method for automatically determining and predicting appropriate staffing or coverage levels (e.g., a staffing or coverage plan) in an enterprise, such as a healthcare facility, based on a number of selected input parameters. The input parameters can include, for example, historical staffing or coverage data, historical patient volume data, current patient volume data, acuity level data, default schedule data, cost by provider type information, contracted staffing or coverage obligations, facility type and size, RVU related information, simulation data, the number of patients seen or expected to be seen during a selected period of time (e.g., patient volume), and the like.

As used herein, the term “enterprise” is intended to include any type of company, institution, facility, or organization that has employees and includes one or more physical plants. The enterprise can include, according to one example, a healthcare facility.

As used herein, the term “healthcare facility” is intended to include hospitals, clinics, facilities rendering medical and healthcare related services, clinician offices, and the like.

As used herein, the term “acuity level” is intended to mean an indication of severity of a medical or healthcare related condition or issue of a patient using any selected type of identifying characteristic, nomenclature, alphanumeric indicator, color coded indicator, and the like. According to one example, the acuity level can include the five-level emergency severity index (ESI) developed by the Agency for Healthcare Research & Quality that is employed by healthcare facilities and which provides a clinically relevant stratification of a patients' conditions into five specific groups—from the most to the least urgent. The index includes a Level 1 designation indicating a patient has a life—threatening condition or issue, a Level 2 designation indicating an emergent/high-risk level condition or issue, a Level 3 designation indicating that the patient has an urgent condition or issue, a Level 4 designation indicating that the patient has a less urgent condition or issue, and a Level 5 designation indicating that the patient has a nonurgent condition or issue. According to another example, any selected indication of severity, such as a numeric score, can be used.

As used herein, the term “staffing plan” or “coverage plan” or is intended to include any type of plan or schedule that can be used to determine or identify current staffing or coverage levels, forecast future staffing or coverage needs, and if desired identify any gaps between the two. The plan or schedule is intended to help enterprises identify and then schedule or acquire the workers or staff that are needed in one or more departments or portions of the enterprise. The staffing plan can encompass the entire enterprise or apply to smaller teams or departments and even individual projects within the enterprise.

As used herein, the term “relative value unit” (RVU) is intended to define the value of a service or procedure relative to all services and procedures rendered by the institution. In a healthcare facility context, this measure of value is based on the extent of physician work, clinical and nonclinical resources, and expertise required to deliver a selected healthcare service to a patient. The RVUs can form part of a general formula that is related to or can assist in determining physician compensation. The RVU is a basic component of the Resource-Based Relative Value Scale (RBRVS), which is a methodology used by the Centers for Medicare & Medicaid Services (CMS) and private payers to determine physician payment.

As used herein, the term “healthcare related data” or “health-related data” is intended to mean or include any structured or unstructured information, records, measurements, or observations generated, collected, processed, or transmitted from individuals, medical devices, healthcare providers, or any other data sources, which pertain or relate to the physical, mental, or emotional well-being of an individual or patient, and which can be received, transmitted, or shared within a healthcare based ecosystem. The healthcare related data encompasses diverse data types and formats relevant to the provision, management, and improvement of healthcare services, patient outcomes, and medical research. The healthcare related data can also encompass a broad range of information, including but not limited to personal health information (PHI), biometric data, medical imaging and diagnostic data, electronic health record (EHR) data, behavioral and/or emotional data, health research data, treatment related data, health administration and billing data, patient provided data, population health data, healthcare analytics and insight related data, and the like.

The personal health information can include data related to an individual's health status, medical history, treatments, medications, allergies, genetic information, and other health-related characteristics. The biometric data can be obtained from various medical devices or wearable technology, and can include as heart rate data, blood pressure data, body temperature data, blood glucose level data, respiratory rate data, and other physiological metric data. The medical imaging and diagnostics data can be received from medical imaging devices, such as X-rays, MRI scans, CT scans, ultrasounds, and other diagnostic procedures used to visualize internal structures for medical assessment. The electronic health records are typically comprehensive digital records containing an individual's health and medical information maintained by one or more healthcare institutions or providers. The behavior data can include information on an individual's lifestyle habits, physical activities, dietary patterns, sleep patterns, and other behavioral factors that impact health. The health research data can include data generated from medical research studies, clinical trials, or population health studies that contribute to the advancement of medical knowledge. The treatment related data can include data related to medical procedures, surgeries, therapeutic interventions, and medication administration. The health administrative and billing data can include information associated with healthcare service utilization, billing, insurance claims, and reimbursement processes. The patient provided data can include information provided by patients themselves, including medical history, symptoms, treatment adherence, quality of life assessments, and patient satisfaction feedback. The population health data can include aggregated data pertaining or relating to the health status, demographics, and health behaviors of specific patient populations or communities. The healthcare analytics and insights related data can include analyzed and processed data that is often in the form of statistical summaries, predictive models, or data visualizations, and which is used to gain actionable insights and support evidence-based decision-making in healthcare. The healthcare data can also include historical and current patient volume data, acuity level data, expected patient throughput data, data associated with a configuration or layout of one or more departments or locations of the healthcare facility, RVU related data, default schedule data, coverage plan data, cost by provider type information, and the like.

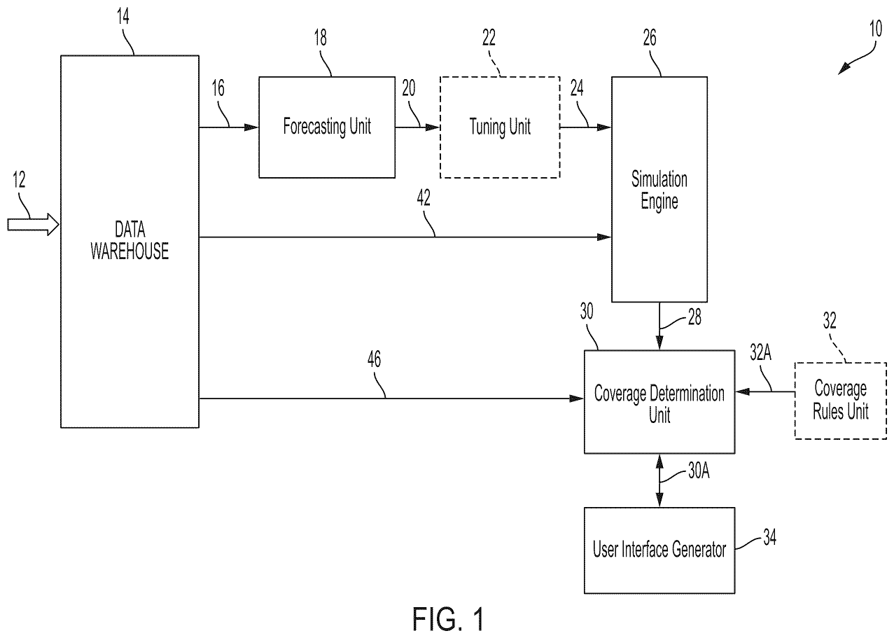

The coverage determination system 10 of the present invention helps determine coverage or staffing needs in an enterprise, such as a healthcare facility, based on selected input parameters to create or generate an appropriate staffing or coverage plan and associated schedule. An example of the coverage determination system 10 of the present invention is shown for example in FIG. 1. The illustrated coverage determination system 10 can include a data storage element 14, such as a data warehouse or data lake that can be configured for receiving, collecting or aggregating together selected types of healthcare related input data 12 associated with the healthcare facility. The healthcare related input data can include, for example, one or more of historical patient volume data, current patient volume data, acuity level data, expected patient throughput data, data associated with a configuration or layout of one or more departments or locations of the healthcare facility, RVU related data, default schedule data, cost by provider type information, and the like. The historical patient volume data 16 can be extracted from a data storage unit, such as the data warehouse 14, such as by using an extract, transform and load (ETL) process, and is then conveyed to a forecasting unit 18. The forecasting unit 18 or the overall system can employ an electronic device having a processor or processing unit that can generate or apply one or more machine learning forecasting models or techniques to the input historical patient volume data 16 to generate staffing or coverage level recommendation data 20, such as for example a patient volume forecast. The forecasting technique can include any suitable machine learning forecasting model or technique, such as for example Facebook (FB) Prophet, an autoregressive integrated moving average (ARIMA) model, and the like. As is known, for example, FB Prophet is a time series forecasting technique and associated model designed for automatic forecasting of univariate time series input data, and especially data that has seasonality aspects (e.g., cyclical patterns) associated therewith. The model associated with the forecasting technique can be trained and retrained using one or more types of the foregoing healthcare related data, including for example historical patient volume data, as well as from the data output of the model. Other types of healthcare related data that can be used for training the forecasting model include current patient volume data, acuity level data, expected patient throughput data, data associated with a configuration or layout of one or more departments or locations of the healthcare facility, RVU related data, default schedule data, cost by provider type information, and the like.

The patient volume forecast data 20 generated by the forecasting model of the forecasting unit 18 can be tuned using an optional tuning unit 22. The tuning unit 22 allows a user or the system to adjust one or more hyperparameters of the underlying machine learning model (e.g., forecasting model) using any selected type of method or technique, such as by using an evolutionary operations technique (EVOP) so as to better reflect and capture changes in the underlying input healthcare related data and to optimize (e.g., Bayesian optimization) the forecast recommendations generated by the forecasting unit 18 for optimum accuracy. The hyperparameters refer to one or more configurable parameters or variables used in the creation and optimization of machine learning models to help determine the behavior, performance, and generalization capabilities of the model. The hyperparameters are distinct from the internal parameters of the model, which are learned during the training process from the provided healthcare related data. Instead, hyperparameters can be set prior to the model training and act or function as tuning knobs that control various aspects of the model, thereby shaping the model's architecture and overall behavior, either during training or thereafter. The hyperparameters can be defined before the training process and remain constant throughout the training iterations or can be varied or changed at selected intervals after the model has been trained. Examples of suitable hyperparameters in the context of machine learning and forecasting models can include the size of the time window or sequence length used as an input to the model so as to define how many past time steps the model considers when making predictions; the number of future time steps the model is trained to predict; learning rate that determines the rate at which the model's parameters are updated during the training process; a number of hidden layers that define the depth of any associated neural network architecture by specifying the number of layers between the input and output layers; the number of neurons per layer that specify the number of neurons (nodes) within each hidden layer of a neural network; batch size for determining the number of training examples used in each iteration (mini-batch) during any associated stochastic gradient descent optimization; regularization strength for controlling any penalty applied to the model's complexity during training to prevent overfitting; a drop out rate to prevent overfitting; seasonality and trend detection related hyperparameters for detecting and incorporating seasonality and trend patterns in the forecasting model; backward and forward lag hyperparameters specifying the number of time steps used as input for lag features (e.g., autoregressive features) in the model; activation functions for defining the mathematical functions applied to the output of any neurons in any associated neural network (if present), thus affecting the model's ability to capture non-linear relationships; dropout rate for specifying the rate at which any associated neurons are randomly deactivated during training, preventing overreliance on specific neurons; and kernel size which can be used in any associated convolutional neural network to define the dimensions of the convolutional filter. Further, the EVOP technique can help improve a process through systematic changes in one or more operating conditions of a given set of factors.

The tuning unit 22 can also employ any suitable tuning technique, such as for example a machine learning R model-based optimization (mlrMBO) technique, also referred to as Bayesian optimization. The mlrMBO technique is a highly configurable model-based/Bayesian optimization modelling of black box functions. The tuning technique (e.g., Bayesian optimization) can be applied to the hyperparameters of the forecasting model. The tuning technique can assist in determining the best or suitable combination of hyperparameters that minimizes or maximizes one or more objective functions of the model, while minimizing the number of function evaluations. The tuning technique initially assists in defining one or more objective functions that are selected to be optimized. In the context of the current hyperparameter tuning technique, this function can represent a performance metric (e.g., accuracy, loss, validation error) of the machine learning model on a given dataset and set of hyperparameters. The tuning technique can also employ a probabilistic model, often a Gaussian Process (GP) model, to model the objective function. The GP model can act as a prior distribution that captures belief about the unknown objective function based on initial function evaluations. The healthcare related data can be employed to fit the Gaussian Process surrogate model. The GP model provides a probabilistic representation of the objective function and its uncertainty. The tuning technique can also select an acquisition function that guides the selection of the next hyperparameter set to evaluate by balancing exploration (e.g., sampling in regions with high uncertainty) and exploitation (e.g., sampling where the objective function is expected to be optimal). Common acquisition functions include probability of improvement (PI), expected improvement (EI), and upper confidence bound (UCB). The acquisition function can be optimized to find the next set of hyperparameters to evaluate, which involves searching for the maximum of the acquisition function. The selected hyperparameter set can be evaluated on the actual objective function to obtain a true objective value, and the new data point can be added to the existing dataset and the GP model updated to incorporate the new information. The tuning unit 22 can generate tuned patient volume forecast data 24, which includes discrete entity data at discrete time periods, that is subsequently conveyed to a simulation engine or unit 26. The tuned patient volume forecast data 24 can include a patient volume forecast that covers any selected time period, such as, in the illustrated example, 60-90 days.

The illustrated simulation engine 26 can simulate for the user or for the system, in any selected manner, one or more business processes as a function of the input data. The simulation engine can be a computational tool designed to model and emulate the behavior of a real-world system or process or enterprise over time. The simulation engine allows the user to understand, analyze, and predict the dynamics of a complex system that may be difficult, costly, or impractical to study directly in the physical world. The simulation engine can model and emulate the behavior of a real-world system or process over time, thus allowing the user the ability to understand, analyze, and predict the dynamics of the complex system. In the current example, the tuned patient volume forecast data 24 can be processed by the simulation engine so that the user can review, visually or otherwise, the output simulation data 28 of the simulation engine 26. The output simulation data 28 can be any selected type of representation, such as a visual representation, of the operation of an existing or proposed system, such as for example the coverage or staffing needs of one or more portions or departments of the healthcare facility, such as an emergency room. As such, the coverage determination system 10 can help plan, manage, and optimize coverage in the healthcare facility based on forecasted or expected patient volume, forecasted patient arrival patterns, and the like. The patient arrival patterns and acuity level data 42 and the patient volume forecast data 24 allows the system to determine or forecast how long patients remain in the emergency room, the locations within the room, the volume of patients, the acuity levels, and the like. One example of a suitable simulation modelling application or technique employed by the simulation engine 26 is the SIMUL8 application manufactured by SIMUL8 Corporation, USA. The simulation engine 26 generates output simulation data 28 that can be subsequently processed by a coverage determination unit 30. The simulation engine 26 can convert or transform the tuned patient volume forecast data 24 over a selected period of time into selected smaller and more discrete time interval increments, such as for example, 30-minute time intervals, based on the tuned patient volume forecast data 24, as well as based on other types of input data or parameters. For example, the additional input data processed by the simulation engine 26 can include acuity level data 42 from the data warehouse 14 about patients that typically frequent the specific healthcare facility or portion of the healthcare facility (e.g., the type of conditions that patients are typically experiencing that visit the healthcare facility). The additional acuity level data 42 can be retrieved, for example, from the data warehouse 14. The simulation engine 26 can also optionally receive layout or configuration data about the department or facility. The lay-out data can be stored in the data warehouse or can be provided separately from a different data source or from the data warehouse 14. Based on the tuned patient volume forecast data 24 and the additional input data 42 (e.g., acuity level data and/or lay-out data), the simulation engine 26 generates the output simulation data 28 representative of these types of data.

The illustrated coverage determination unit 30 receives and processes the output simulation data 28, and optionally additional data, to generate a staffing or coverage plan. The additional data can include selected types of input healthcare related data 46 received from the data warehouse 14. The additional input or healthcare related data 46 can include coverage data including one or more or any selected combination of default schedule data, contract staffing requirement data, relative value unit capacity data, types of service providers or clinicians (e.g., doctors, nurses, physician assistants, and the like), cost by provider type data, additional acuity level data, predefined staffing requirements based on the acuity level (e.g., what type of clinicians to schedule based on the acuity level), contract staffing rules or requirement data, patient treatment related data, and the like. The contract staffing rules or requirement data typically includes information associated with required or contracted staffing levels, which need to be considered when generating the coverage plan or schedule. The coverage determination system 10 can also include an optional coverage rules unit 32 that has prestored therein selected types of rules-based information. In the current example, the coverage rules-based information can include information associated with a length of the coverage shifts (e.g., no shift less than 8 hours), time period limitations (e.g., no shift starts between 12 AM-5 AM), predetermined or contracted number of shifts per location or site, and the like. The coverage rules unit 32 can provide the coverage rules data 32A to the coverage determination unit 30. The coverage determination unit 30 can be a separate unit, as shown, or can form part of the data warehouse 14, or form part of the coverage determination unit 30. The coverage determination unit 30 then processes the optional input or healthcare related data 46, the optional coverage rules information 32A, and the output simulation data 28, and generates in response thereto and based thereon a staffing or coverage plan and associated data 30A. Specifically, the coverage determination unit processes all of the incoming data and then matches staff coverage needs in any selected time increments, such as for example in 30-minute increments, for a selected period of time, such as for example for each day or for a month, of the healthcare facility. The generated coverage plan data 30A is in essence a schedule that recommends selected coverage based on a number of different factors including coverage needs and costs per shift for each facility. As such, the coverage determination unit 30 optimizes staff coverage and cost. In the healthcare facility context, the staff or clinicians can include doctors, nurses, nurse practitioners, physician assistants, assistants, therapists, and other types of healthcare workers or employees.

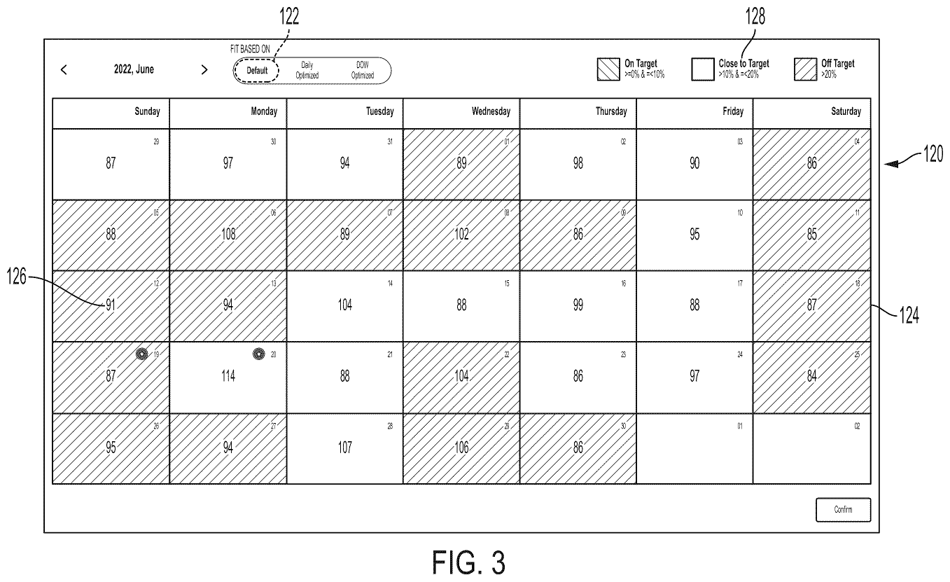

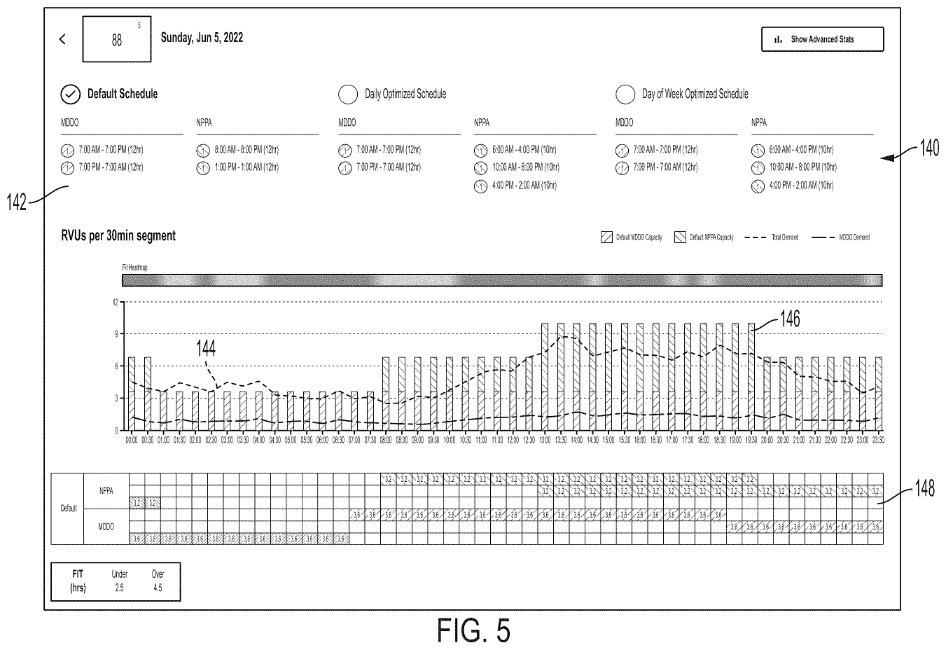

The illustrated coverage plan data 30A can be conveyed to a user interface generator 34 that generates one or more user interfaces for displaying the coverage plan generated for each healthcare facility. Examples of user interfaces that can be generated by the user interface generator 34 are shown in FIGS. 3-9. FIG. 3, for example, illustrates an initial user interface or window element 120 (herein generally referred to as a window, a frame or a page) generated by the user interface generator 34. The illustrated window 120 and the associated data can be displayed on a suitable display device, such as the display of a suitable electronic device, such as a computer or a smart phone. The window 120 has a main area formed therein that has a calendar of any selected time period, such as a day, week, or month. The calendar can be populated by the coverage determination unit 30 to form a default schedule or coverage plan 124 covering a selected period of time, such as a month. The month view is the default view and can be selected by the user actuating a soft button 122. The schedule 124 can be illustrated in any selected form or format, and preferably can be shown in a calendar view format. The schedule 124 can show any selected type of information, such as for example the patient volume 126 (as shown) per day or number of staff per day, or both. The schedule can be selectively color coded 128 to form in essence a heat map to convey selected types of information, such as whether there is adequate coverage for a selected day or the anticipated or expected patient volume. The window 120 thus assists the operations team in evaluating the efficiency and effectiveness of the schedule or coverage plan 124 in meeting demand across a selected period of time (e.g., a month).

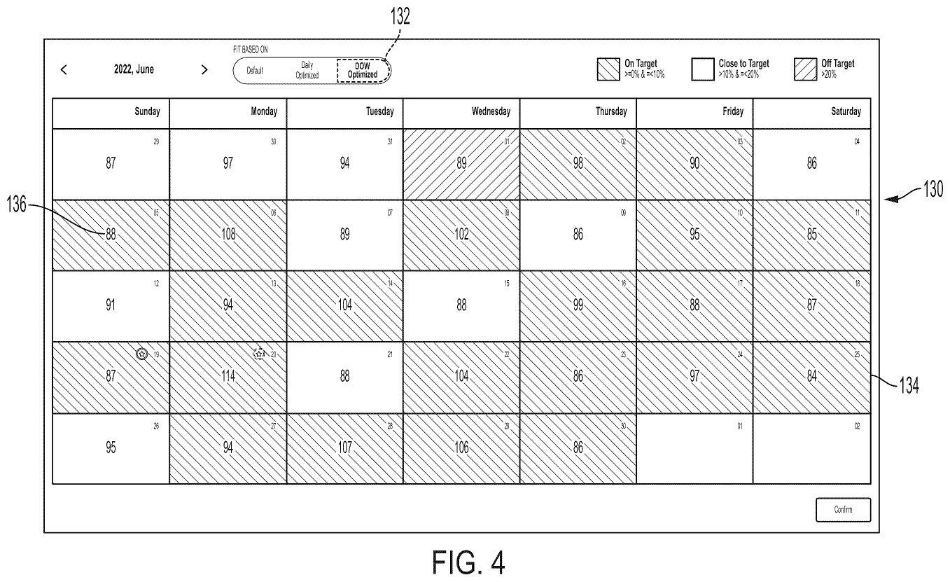

FIG. 4 shows a window 130 generated by the user interface generator 34 when the optimized soft button 132 is selected or actuated by a user. The window 130 illustrates the optimized coverage plan or schedule 134 in the same calendar view format of FIG. 3, and the coverage for each day in the calendar can be optimized by the coverage determination system 10 and displayed in the calendar format. As clearly shown in the illustrated window 130, the coverage fit based on anticipated or expected patient volume 136 for each day is optimized relative to the default schedule 124. The days in the calendar can be selected by the user to view additional information. For example, if the June 05 day in the window 130 is selected, the user interface generator 34 can generate an additional user interface or window 140, as shown in FIG. The illustrated window 140 can include a series of panes, such as the vertically stacked set of panes, for displaying various types of information. The illustrated window can include an uppermost pane that displays information associated with one or more of a default schedule or a coverage plan generated by the coverage determination unit 30. The schedule information can be displayed in any selected format and cover any selected time frame, such as in the illustrated daily time period format. The window 140 can also display in a different pane element selected coverage information, including a range of coverage information, as well as selected RVU information. The dashed lines 144 are indicative of the anticipated patient demand and the stacked bars 146 are indicative of the clinician staffing (e.g., supply) in RVUs. The window 140 can also include in a bottom-most pane element for displaying staffing information 148 that can be displayed in any selected format. As shown, the staffing information includes the number of healthcare workers or staff by selected categories, such as nurses and doctors.

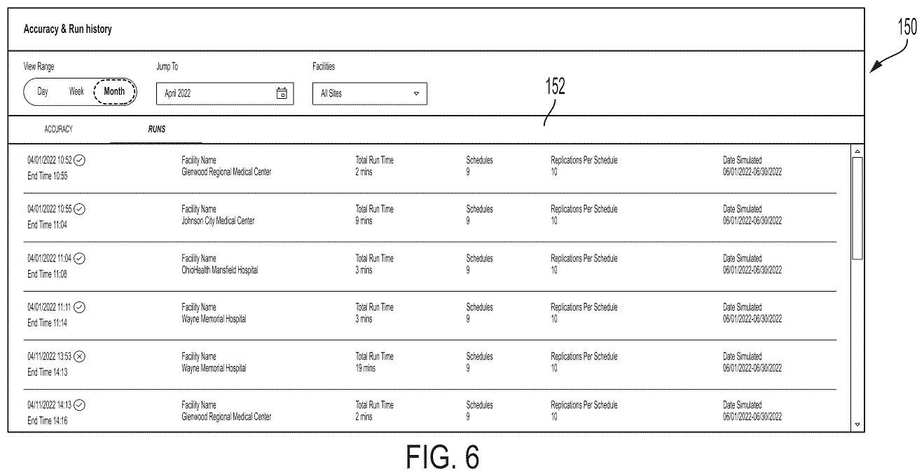

FIG. 6 illustrates a user interface or window 150 generated by the user interface generator 34 if the user wishes to display and hence review the run history 152 of the information generated by the coverage determination system 10 of the present invention. The system administrator uses the run history 152 to monitor the system in case there is a data problem.

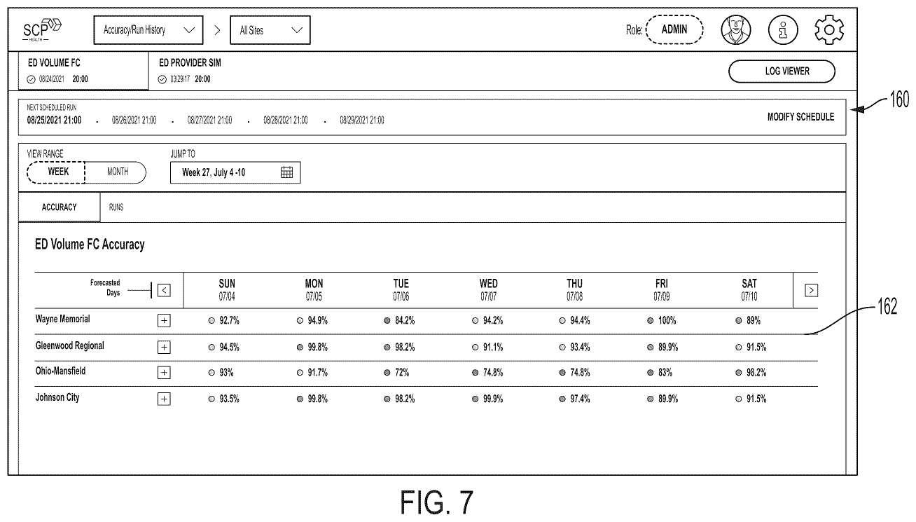

FIG. 7 is a schematic representation of a user interface or window 160 generated by the user interface generator 34 of the present invention. The user interface generator 34 can generate the illustrated widow 160 when the user actuates selected soft buttons to display forecast accuracy of the coverage plan generated by the coverage determination unit. In the current example, the window 160 displays a forecast accuracy dashboard 162 of the emergency department of one or more selected healthcare facilities. The dashboard 162 can include any selected type of information, such as the specific facility, time or day of month or year, and the forecast accuracy of the coverage plan. The forecast accuracy can be expressed in any selected manner, such as a percentage. The accuracy percentages can be color coded to reflect a patient volume forecast accuracy within a selected accuracy range or relative to one or more accuracy threshold values and/or to reflect a staffing level forecast accuracy within a selected accuracy range or relative to one or more accuracy threshold values. The illustrated dashboard 162 can be used by a system administrator to track the accuracy of the patient volume forecast or the staff coverage accuracy, which is a key input to the simulation engine. If the forecast accuracy starts to change or drift relative to a threshold value or relative to expected values, the administrator can trigger the tuning unit 22 to reset one or more of the hyperparameters of the forecasting model of the forecasting unit 18.

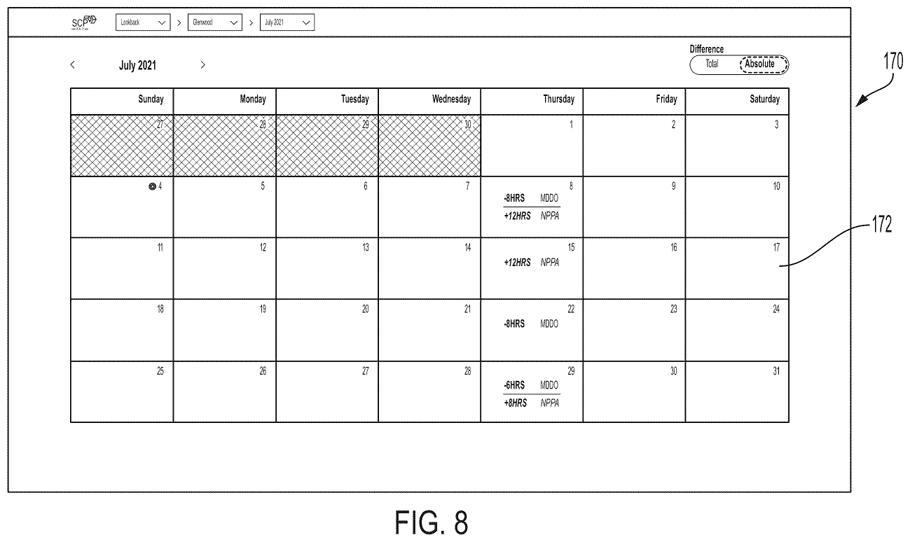

FIG. 8 is a schematic representation of a user interface or window 170 generated by the user interface generator 34 of the present invention. The user interface generator 34 can generate the illustrated window 170 when the user actuates selected soft buttons to display past coverage (e.g., lookback) at a selected healthcare facility. In the current example, the user interface 170 shows a calendar view 172 that illustrates or displays the coverage for selected days, including the type of healthcare staff covering selected shifts during the days. The staff can include nurse practitioners, physician assistants, medical doctors, and the like. The healthcare staff can be broken down by category and displayed as such in the interface, if desired. Further, the window 170 can show the actual staffing team versus what the user selected using the system.

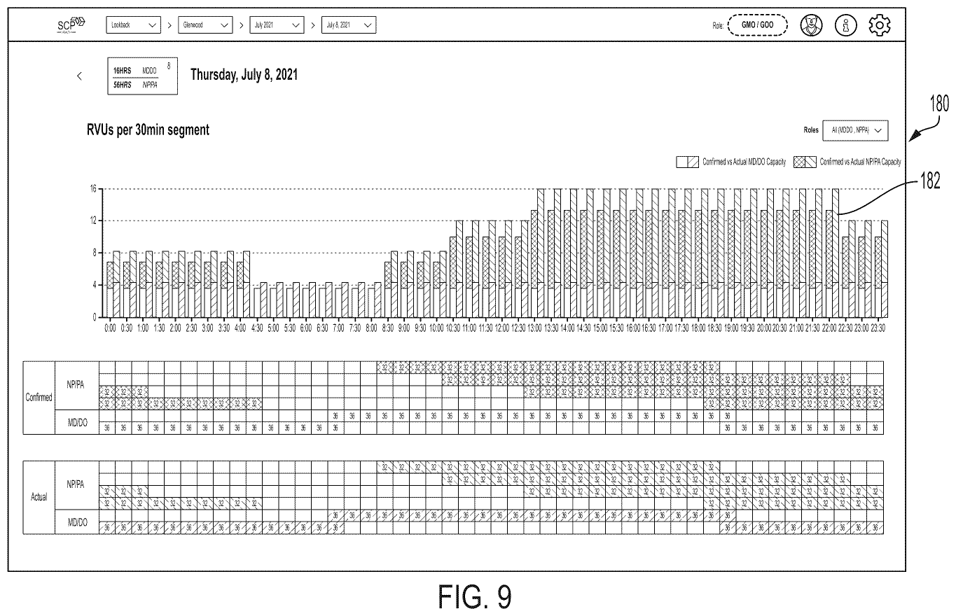

FIG. 9 is a schematic representation of a user interface or window 180 generated by the user interface generator 34 of the present invention. The user interface generator 34 can generate the illustrated window 180 when the user actuates one or more of the days in the user interface 170 shown in FIG. 8 in order to drill down in the information associated with the selected day. In the current example, the user interface 180 displays the staffing coverage 182 in RVUs associated with each category or class of healthcare staffer for the selected day. The information can be broken down according to selected time increments, such as 30 minutes. The user interface 180 also displays the comparison between the actual observed demand versus the actual staffed capacity (in RVUs).

With reference again to FIG. 1, the user can review the resulting or generated schedule or coverage plan 30A and modify the coverage plan or modify any of the selected parameters processed by the coverage determination unit 30, such as for example implementing changes to the coverage rules and the like so as to tune or modify the coverage plan 30A. The user interfaces display to the user the suggested coverage plan 30A, and the coverage plan can be displayed simultaneously with other types of information, such as for example anticipated patient volume in RVUs. The user can also fine tune or modify selected parameters of the coverage plan, such as for example a certain day on the calendar until the desired information is achieved or simultaneously solve for issues on certain days of the week or for the whole month. This allows for more consistent work schedules and significantly reduces worker stress. The user (e.g., schedulers) can then review the resulting schedules and then decide to approve the schedules or modify the schedule. The resulting approved schedules can then be imported into a scheduling system or manually adjusted before being entered into a scheduling system of the healthcare facility.

The coverage determination system 10 of the present invention can thus automatically generate, based on selected types of input data, a coverage plan 30A that provides optimal staffing coverage, including by numbers and healthcare staff types (e.g., medical doctors as compared to nurse practitioners) in response to a simulated environment of the healthcare facility. The coverage plan 30A can include additional information, such as RVU information and acuity demand information in selected time increments (e.g., 30-minute time increments). The user interface generator 34 can also generate user interfaces for displaying current and optimal coverage plans.

The coverage determination system 10 can also generate coverage plans for additional healthcare facilities that are similar to the facilities that the system has stored historical data based on an automatic selection of a ‘like site’ feature, which is the next closest facility in terms of urban vs rural, geographical location, and patient volume.

The advantages of the coverage determination system 10 of the present invention can include the ability to automatically solve for and generate the most efficient provider schedule via an automatically generated coverage plan. By generating an optimal coverage plan, the coverage determination system 10 provides for significant cost savings compared to prior art techniques for creating coverage plans. The ability generate an optimized coverage plan results in increased provider satisfaction and improved productivity with less wait time for patients. The coverage plan 30A generated by the present invention also helps to proactively align coverage in selected time increments, resulting in increased satisfaction for the healthcare staffers.

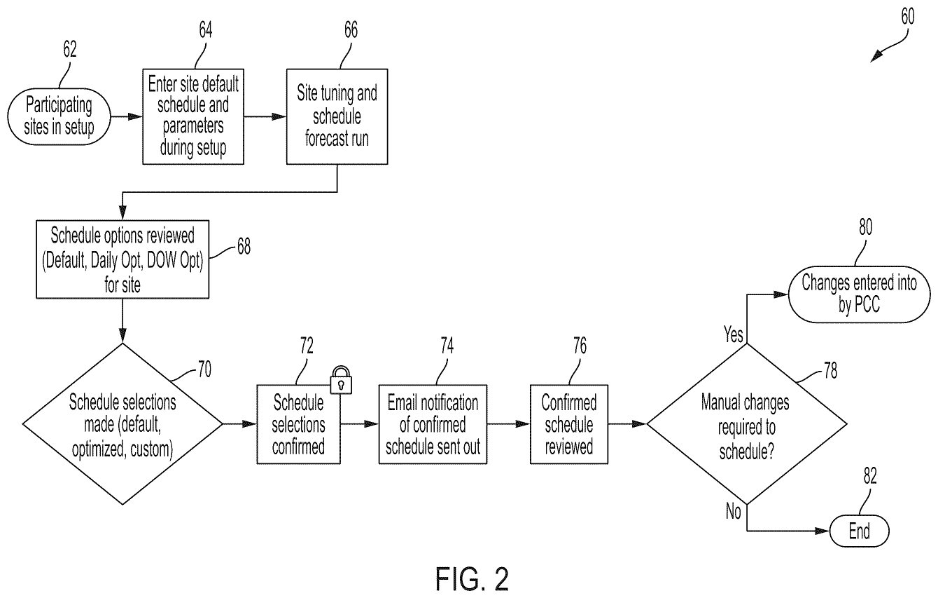

FIG. 2 is a schematic flow chart diagram 60 illustrating the method for generating an optimized coverage plan according to the teachings of the present invention. The flow chart 60 allows selected healthcare facilities to link their scheduling systems to the coverage determination system 10 of the present invention, step 62. The data warehouse 14 receives input data 12 from the healthcare facility. The input data can include, as previously noted, historical patient volume data, prior coverage plans, and the like, step 64. The coverage determination unit can generate a coverage plan 30A based on selected types of input data, including output simulation data, acuity level data, patient volume throughput data, coverage rules data, and the like, step 66. The user through the selected user interfaces generated by the user interface generator 34 can review the coverage plan 30A and selected data in the coverage plan, step 68. The user can confirm the coverage plan 30A generated by the coverage determination unit 30 or can modify the generated coverage plan, step 70. The user then accepts the final coverage plan, step 72. The coverage determination system 10 of the present invention can integrate with the scheduling system of the healthcare facility, and any selected type of communication, such as an electronic communication (e.g., e-mail), can be auto generated and the coverage plan can be sent to the staff, step 74. The system can be configured to await verification from each staff member that they received and reviewed the coverage plan, step 76. The staff can provide information relative to the coverage plan 30A, and additional changes can be made, if desired, step 78. If further changes are needed, the user can enter the changes into the system so that an updated coverage plan 30A is generated, step 80. Otherwise, the coverage plan is deemed to be final, step 82.

It is to be understood that although the invention has been described above in terms of particular embodiments, the foregoing embodiments are provided as illustrative only and do not limit or define the scope of the invention. Various other embodiments, including but not limited to those described herein, are also within the scope of the claims. For example, elements, units, tools and components described herein may be further divided into additional components or joined together to form fewer components for performing the same functions.

Any of the functions disclosed herein may be implemented using means for performing those functions. Such means include, but are not limited to, any of the components or units disclosed herein, such as the electronic or computing device components described herein.

The techniques described above and below may be implemented, for example, in hardware, one or more computer programs tangibly stored on one or more computer-readable media, firmware, or any combination thereof. The techniques described above may be implemented in one or more computer programs executing on (or executable by) a programmable computer or electronic device having any combination of any number of the following: a processor, a storage medium readable and/or writable by the processor (including, for example, volatile and non-volatile memory and/or storage elements), an input device, an output device, and a display. Program code may be applied to input entered using the input device to perform the functions described and to generate output using the output device.

The term computer, computing device or electronic device as used herein can refer to any device that includes a processor and a computer-readable memory capable of storing computer-readable instructions, and in which the processor is capable of executing the computer-readable instructions in the memory. The terms computer system and computing system refer herein to a system containing one or more computing devices.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention may operate on digital electronic processes which can only be created, stored, modified, processed, and transmitted by computing devices and other electronic devices. Such embodiments, therefore, address problems which are inherently computer-related and solve such problems using computer technology in ways which cannot be solved manually or mentally by humans.

Any claims herein which affirmatively require a computer, an electronic device, a processor, a memory, storage, or similar computer-related elements, are intended to require such elements, and should not be interpreted as if such elements are not present in or required by such claims. Such claims are not intended, and should not be interpreted, to cover methods and/or systems which lack the recited computer-related elements. For example, any system or method claims herein which recite that the claimed method is performed by a computer, a processor, a memory, and/or similar computer-related element, is intended to, and should only be interpreted to, encompass systems and methods which are performed by the recited computer-related element(s). Such a system and method claim should not be interpreted, for example, to encompass a system or method that is performed mentally or by hand (e.g., using pencil and paper). Similarly, any product or computer readable medium claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Embodiments of the present invention solve one or more problems that are inherently rooted in computer technology. For example, embodiments of the present invention solve the problem of how to generate or determine a coverage plan for an enterprise. There is no analog to this problem in the non-computer environment, nor is there an analog to the solutions disclosed herein in the non-computer environment.

Furthermore, embodiments of the present invention represent improvements to computer and communication technology itself. For example, the system 10 of the present can optionally employ a specially programmed or special purpose computer in an improved computer system, which may, for example, be implemented within a single computing device. Further, the system of the present invention can be configured or implemented using one or more computing or electronic devices as described herein, such as a server, a computer, a processor, a memory, storage, and the like.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements can also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

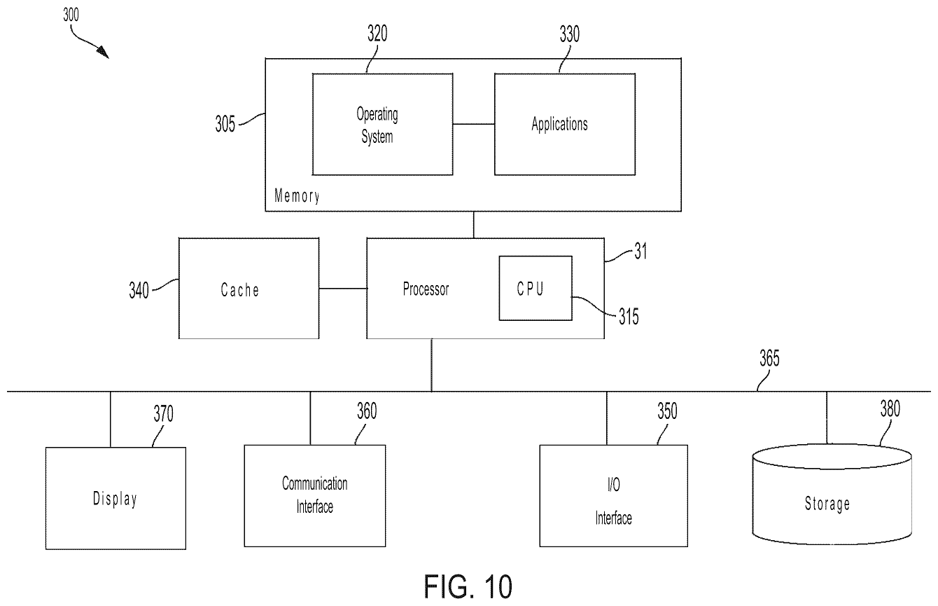

It should be appreciated that various concepts, systems, and methods described above can be implemented in any number of ways, as the disclosed concepts are not limited to any particular manner of implementation or system configuration. Examples of specific implementations and applications are discussed below and shown in FIG. 10 primarily for illustrative purposes and for providing or describing the operating environment of the system of the present invention. The system 10 and/or elements or units or engines thereof can employ one or more electronic or computing devices, such as one or more servers, clients, computers, laptops, smartphones and the like, that are networked together, or which are arranged so as to effectively communicate with each other. The network can be any type or form of network. The devices can be on the same network or on different networks. In some embodiments, the network system may include multiple, logically grouped servers. In one of these embodiments, the logical group of servers may be referred to as a server farm or a machine farm. In another of these embodiments, the servers may be geographically dispersed. The electronic devices can communicate through wired connections or through wireless connections. The clients can also be generally referred to as local machines, clients, client nodes, client machines, client computers, client devices, endpoints, or endpoint nodes. The servers can also be referred to herein as servers, server nodes, or remote machines. In some embodiments, a client has the capacity to function as both a client or client node seeking access to resources provided by a server or server node and as a server providing access to hosted resources for other clients. The clients can be any suitable electronic or computing device, including for example, a computer, a server, a smartphone, a smart electronic pad, a portable computer, and the like, such as the illustrated electronic or computing device 300. The system 10 or any associated units or components of the system 10 can employ one or more of the illustrated computing devices and can form a computing system. Further, the server may be a file server, application server, web server, proxy server, appliance, network appliance, gateway, gateway server, virtualization server, deployment server, SSL VPN server, or firewall, or any other suitable electronic or computing device, such as the electronic device 300. In one embodiment, the server may be referred to as a remote machine or a node. In another embodiment, a plurality of nodes may be in the path between any two communicating servers or clients. The coverage determination system 10 which includes the forecasting unit, the tuning unit, the simulation engine, the coverage determination unit, the coverage rules unit, and the interface generator can be stored on or implemented by one or more of the electronic or computing devices described herein (e.g., clients or servers), and the hardware associated with the electronic devices, such as the processor or CPU and memory described below.

FIG. 10 is a high-level block diagram of an electronic or computing device 300 that can be used with the embodiments disclosed herein. Without limitation, the hardware, software, and techniques described herein can be implemented in digital electronic circuitry or in computer hardware that executes firmware, software, or combinations thereof. The implementation can include a computer program product (e.g., a non-transitory computer program tangibly embodied in a machine-readable storage device, for execution by, or to control the operation of, one or more data processing apparatuses, such as a programmable processor, one or more computers, one or more servers and the like).

The illustrated electronic device 300 can be any suitable electronic circuitry that includes a main memory unit 305 that is connected to a processor 311 having a CPU 315 and a cache unit 340 configured to store copies of the data from the most frequently used main memory 305. The electronic device can implement the process flow identification system 10 or one or more elements of the process flow identification system.

Further, the methods and procedures for carrying out the methods disclosed herein can be performed by one or more programmable processors executing a computer program to perform functions of the invention by operating on input data and generating output. Further, the methods and procedures disclosed herein can also be performed by, and the apparatus disclosed herein can be implemented as, special purpose logic circuitry, such as a FPGA (field programmable gate array) or an ASIC (application specific integrated circuit). Modules and units disclosed herein can also refer to portions of the computer program and/or the processor/special circuitry that implements that functionality.

The processor 311 is any logic circuitry that responds to, processes or manipulates instructions received from the main memory unit, and can be any suitable processor for execution of a computer program. For example, the processor 311 can be a general and/or special purpose microprocessor and/or a processor of a digital computer. The CPU 315 can be any suitable processing unit known in the art. For example, the CPU 315 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively, or additionally, the processor can comprise at least one of a multi-core processor and a front-end processor. Generally, the processor 311 can be embodied in any suitable manner. For example, the processor 311 can be embodied as various processing means such as a microprocessor or other processing element, a coprocessor, a controller or various other computing or processing devices including integrated circuits such as, for example, an ASIC (application specific integrated circuit), an FPGA (field programmable gate array), a hardware accelerator, or the like. Additionally, or alternatively, the processor 311 can be configured to execute instructions stored in the memory 305 or otherwise accessible to the processor 311. As such, whether configured by hardware or software methods, or by a combination thereof, the processor 311 can represent an entity (e.g., physically embodied in circuitry) capable of performing operations according to embodiments disclosed herein while configured accordingly. Thus, for example, when the processor 311 is embodied as an ASIC, FPGA or the like, the processor 311 can be specifically configured hardware for conducting the operations described herein. Alternatively, as another example, when the processor 311 is embodied as an executor of software instructions, the instructions can specifically configure the processor 311 to perform the operations described herein. In many embodiments, the central processing unit 530 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The processor can be configured to receive and execute instructions received from the main memory 305.

The electronic device 300 applicable to the hardware of the present invention can be based on any of these processors, or any other processor capable of operating as described herein. The central processing unit 315 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

The processor 311 and the CPU 315 can be configured to receive instructions and data from the main memory 305 (e.g., a read-only memory or a random-access memory or both) and execute the instructions. The instructions and other data can be stored in the main memory 305. The processor 311 and the main memory 305 can be included in or supplemented by special purpose logic circuitry. The main memory unit 305 can include one or more memory chips capable of storing data and allowing any storage location to be directly accessed by the processor 311. The main memory unit 305 may be volatile and faster than other memory in the electronic device, or can dynamic random access memory (DRAM) or any variants, including static random access memory (SRAM), Burst SRAM or SynchBurst SRAM (B SRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 305 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 305 can be based on any of the above-described memory chips, or any other available memory chips capable of operating as described herein. In the embodiment shown in FIG. 4, the processor 311 communicates with main memory 305 via a system bus 365. The computer executable instructions of the present invention may be provided using any computer-readable media that is accessible by the computing or electronic device 300. Computer-readable media may include, for example, the computer memory or storage unit 305. The computer storage media may also include, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer readable instructions, data structures, program modules, or other data in a modulated data signal, such as a carrier wave, or other transport mechanism. As defined herein, computer readable storage media does not include communication media. Therefore, a computer storage or memory medium should not be interpreted to be a propagating signal per se or stated another transitory in nature. The propagated signals may be present in a computer storage media, but propagated signals per se are not examples of computer storage media, which is intended to be non-transitory. Although the computer memory or storage unit 305 is shown within the computing device 300 it will be appreciated that the storage may be distributed or located remotely and accessed via a network or other communication link.

The main memory 305 can comprise an operating system 320 that is configured to implement various operating system functions. For example, the operating system 320 can be responsible for controlling access to various devices, memory management, and/or implementing various functions of the asset management system disclosed herein. Generally, the operating system 320 can be any suitable system software that can manage computer hardware and software resources and provide common services for computer programs.

The main memory 305 can also hold application software 330. For example, the main memory 305 and application software 330 can include various computer executable instructions, application software, and data structures, such as computer executable instructions and data structures that implement various aspects of the embodiments described herein. For example, the main memory 305 and application software 330 can include computer executable instructions, application software, and data structures, such as computer executable instructions and data structures that implement various aspects of the content characterization systems disclosed herein, such as processing and capture of information. Generally, the functions performed by the content characterization systems disclosed herein can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers). Generally, the program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 311 can further be coupled to a database or data storage 380. The data storage 380 can be configured to store information and data relating to various functions and operations of the content characterization systems disclosed herein. For example, as detailed above, the data storage 380 can store information including but not limited to captured information, multimedia, processed information, and characterized content.

A wide variety of I/O devices may be present in or connected to the electronic device 300. For example, the electronic device can include a display 370, and as previously described, the visual application unit 28 or one or more other elements of the system 10 can include the display. The display 370 can be configured to display information and instructions received from the processor 311. Further, the display 370 can generally be any suitable display available in the art, for example a Liquid Crystal Display (LCD), a light emitting diode (LED) display, digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays, or electronic papers (e-ink) displays. Furthermore, the display 370 can be a smart and/or touch sensitive display that can receive instructions from a user and forwarded the received information to the processor 311. The input devices can also include user selection devices, such as keyboards, mice, trackpads, trackballs, touchpads, touch mice, multi-touch touchpads, touch mice and the like, as well as microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. The output devices can also include video displays, graphical displays, speakers, headphones, inkjet printers, laser printers, and 3D printers.

The electronic device 300 can also include an Input/Output (I/O) interface 350 that is configured to connect the processor 311 to various interfaces via an input/output (I/O) device interface 380. The device 300 can also include a communications interface 360 that is responsible for providing the circuitry 300 with a connection to a communications network (e.g., communications network 120). Transmission and reception of data and instructions can occur over the communications network.

The electronic or computing device 300 may be designed and/or configured to perform any method, method step, or sequence of method steps in any embodiment described in this disclosure, in any order and with any degree of repetition. For instance, electronic device 300 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The electronic device 300 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

In the present disclosure, data used to train a machine learning model, such as a forecasting model, can include data containing correlations that a machine-learning process or technique may use to model relationships between two or more types or categories of data elements (“training data”). For instance, and without limitation, the training data may include a plurality of data entries, each entry representing a set of data elements that were recorded, received, and/or generated together. The data elements may be correlated by shared existence in a given data entry, by proximity in a given data entry, or the like. Multiple data entries in the training data may evince one or more trends in correlations between categories or types of data elements. For instance, and without limitation, a higher value of a first data element belonging to a first category or types of data element may tend to correlate to a higher value of a second data element belonging to a second category or type of data element, indicating a possible proportional or other mathematical relationship linking values belonging to the two categories. Multiple categories of data elements may be related in training data according to various correlations, and the correlations may indicate causative and/or predictive links between categories of data elements, which may be modeled as relationships such as mathematical relationships by the machine-learning processes as described herein. The training data may be formatted and/or organized by categories of data elements, for example by associating data elements with one or more descriptors corresponding to categories of data elements. As a non-limiting example, training data may include data entered in standardized forms by persons or processes, such that entry of a given data element in a given field in a given form may be mapped or correlated to one or more descriptors of categories. Elements in training data may be linked to descriptors of categories or types by tags, tokens, or other data elements. For example, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively, or additionally, the training data may include one or more data elements that are not categorized, that is, the training data may not be formatted or contain descriptors for some elements of data. Machine-learning models or algorithms and/or other processes may sort the training data according to one or more categorizations using, for instance, natural language processing algorithms, tokenization, detection of correlated values in raw data and the like. The categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name or other types of data may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by the electronic device 300 may correlate any input data as described in this disclosure to any output data as described in this disclosure.

The present invention can employ one or more machine learning models, such as a forecasting model. The forecasting models can be employed to address or handle different types of forecasting tasks. Some of the main types of forecasting machine learning models include autoregressive models that use past observations of a target variable as an input to predict future values (e.g., ARIMA (AutoRegressive Integrated Moving Average)); seasonal autoregressive integrated moving-average (SARIMA) models extend ARIMA by considering seasonal patterns in addition to the regular autoregressive, integrated, and moving average components; exponential smoothing models that use weighted averages of past observations to predict future values and are useful for time series data with no clear trend or seasonality (e.g., Simple Exponential Smoothing, Double Exponential Smoothing (Holt's method), and Triple Exponential Smoothing (Holt-Winters' method)); long short-term memory (LSTM) networks which is a type of recurrent neural network (RNN) capable of learning long-term dependencies in time series data and are particularly effective for processing sequential data with complex temporal patterns; gradient boosting machines (GBM) which is an ensemble learning technique that builds multiple decision trees to make predictions (e.g., XGBoost and LightGBM for time series forecasting); prophet or Facebook Prophet incorporates time series components such as seasonality and holidays and can effectively handle missing data and outliers; neural prophet is an extension of the Facebook Prophet model that utilizes neural networks to capture complex temporal patterns and relationships in the data; DeepAR which is a probabilistic forecasting model based on recurrent neural networks that can handle uncertainty in time series data; VAR (Vector Autoregression) models which can be used for multivariate time series forecasting, where multiple related variables are forecasted simultaneously; and CNN (Convolutional Neural Networks) for Time Series models which can be adapted for time series forecasting, especially when dealing with 1D sequences. The choice or selection of the proper forecasting model depends on the specific characteristics of the time series data, the presence of trends and seasonality, the amount of data available, and the desired level of interpretability and complexity.

The ARIMA (AutoRegressive Integrated Moving Average) model is a time series forecasting technique that can be used to analyze and predict data with temporal dependencies, and can be a combination of three components: AutoRegressive (AR), Integrated (I), and Moving Average (MA). The ARIMA model is capable of capturing both short-term and long-term patterns in a time series dataset. The AR component represents the relationship between the current observation and its past values, and assumes that the current value of the time series can be predicted using its previous values. The “p” parameter in ARIMA (denoted as AR(p)) indicates the number of lagged observations included in the model. For example, AR(1) uses the previous observation, AR(2) uses the two previous observations, and so forth. The Integrated (I) component refers to the process of differencing the time series data to make it stationary. Stationarity implies that the mean, variance, and autocorrelation structure of the time series remain constant over time. Differencing involves computing the differences between consecutive observations to remove trends and make the data stationary. The “d” parameter in ARIMA (denoted as I(d)) represents the order of differencing. The Moving Average (MA) component represents the relationship between the current observation and the residual errors from previous observations, and accounts for the short-term fluctuations that are not explained by the autoregressive components. The “q” parameter in ARIMA (denoted as MA(q)) indicates the number of lagged forecast errors included in the model. The general notation for an ARIMA model is ARIMA(p, d, q), where: p is the order of the AutoRegressive component, d is the order of differencing (number of times the data is differenced to achieve stationarity), and q is the order of the Moving Average component. To apply an ARIMA model to a time series dataset or to training data, the steps can include inspecting the data for seasonality, trends, and stationarity and if necessary applying differencing to make the data stationary; determining the appropriate values of p, d, and q by analyzing autocorrelation and partial autocorrelation plots; fitting the ARIMA model to the transformed data; and then making forecasts and evaluating the model's performance.

The machine learning processes can generate machine learning models that can be categorized into different types based on their learning approaches and characteristics. The machine learning models can include supervised learning models (e.g., linear regression models, logistic regression models, decision tree models, random forest models, support vector models, neural network models), unsupervised learning models (e.g., K-Means clustering models, hierarchical clustering models, principal component analysis (PCA) models, gaussian mixture models (GMM)), semi-supervised learning models (e.g., a combination of supervised and unsupervised learning approaches where the model is trained on a partially labeled dataset), reinforcement learning models (e.g., agents and Q-learning and deep Q networks (DQNs)), transfer learning models (e.g., pretrained models that are used as a starting point for related tasks to improve learning efficiency and performance), ensemble learning models (e.g., models that combine multiple models to make predictions, such as Bagging, Boosting, and Stacking), deep learning models (e.g., neural networks, convolutional neural networks (CNNs), and recurrent neural networks (RNNs)), and time series models (e.g., specialized models designed to analyze and forecast time-dependent data, such as ARIMA, Prophet, and LSTM).

The machine-learning processes as described herein may be used to generate machine-learning models. A machine-learning model, as used herein, is a mathematical representation of a relationship between inputs and outputs, as generated using any machine-learning process including without limitation any process as described above and stored in memory. An input can be submitted to a machine-learning model once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

We claim:

1. A computer implemented coverage determination system for determining a coverage plan for a healthcare facility, comprising
a processor,
a non-transitory memory having instructions configuring the processor to:
train a forecasting model with historical patient volume data as an input and correlating the historical patient volume data with the healthcare facility, wherein the trained forecasting model generates patient volume forecast data in response to the patient volume data, wherein the forecasting model includes a plurality of hyperparameters and the patient volume data covers a patient volume over a first selected period of time,
tuning one or more of the plurality of hyperparameters by applying thereto a tuning technique, wherein the tuning unit generates in response tuned patient volume forecast data,
generating with a simulation engine simulation data in response to the tuned patient volume forecast data and acuity level data, wherein the simulation data includes the patient volume over a second period of time, wherein the second period of time is shorter than the first period of time, and
generating a coverage plan in response to and based on the simulation data, coverage data received from a data storage unit, and coverage rules data from a coverage rules unit.

2. The computer implemented system of claim 1, wherein the simulation engine simulates the coverage plan based on the tuned patient volume forecast data, the acuity level data, and lay-out data of the healthcare facility.

3. The computer implemented system of claim 2, wherein the simulation data includes a visual representation of a coverage requirement of one or more portions of the healthcare facility.

4. The computer implemented system of claim 3, wherein the coverage data comprises one or more of default schedule data, contract staffing requirement data, relative value unit capacity data, service provider type data, cost by provider type data, additional acuity level data, predefined staffing requirements based on the acuity level, contract staffing rules data, and patient treatment related data.

5. The computer implemented system of claim 4, wherein the coverage rules data comprises one or more of a length of a coverage shift, one or more time period limitations on the shift, and a predetermined number of shifts per healthcare facility.

6. The computer implemented system of claim 5, wherein the coverage plan matches staff coverage needs in one or more selected time increments for a selected period of time with the healthcare facility.

7. The computer implemented system of claim 6, wherein the coverage plan further includes one or more coverage recommendations based on coverage needs and costs per shift for the healthcare facility.

8. The computer implemented system of claim 7, further comprising a user interface generator for generating in response to the coverage plan one or more user interfaces for displaying the coverage plan.

9. A coverage determination system for determining a coverage plan for a healthcare facility, comprising
a forecasting unit for applying a forecasting model to patient volume data received from a data storage unit and then generating in response patient volume forecast data, wherein the forecasting model includes a plurality of hyperparameters and the patient volume data covers a patient volume over a first selected period of time,
a tuning unit for tuning one or more of the plurality of hyperparameters by applying thereto a tuning technique, wherein the tuning unit generates in response tuned patient volume forecast data,
a simulation engine for receiving the tuned patient volume forecast data and acuity level data and then generating in response thereto simulation data, wherein the simulation data includes the patient volume over a second period of time, wherein the second period of time is shorter than the first period of time, and
a coverage determination unit for receiving the simulation data, coverage data from the data storage unit, and coverage rules data from a coverage rules unit, and then generating in response thereto the coverage plan.

10. The computer implemented system of claim 9, wherein the learning forecasting model comprises Facebook Prophet or ARIMA.

11. The computer implemented system of claim 10, wherein the tuning technique includes a machine learning R model based optimization (mlrMBO) technique.

12. The computer implemented system of claim 11, wherein the simulation engine simulates the coverage plan based on the tuned patient volume forecast data, the acuity level data, and lay-out data of the healthcare facility.

13. The computer implemented system of claim 12, wherein the simulation data includes a visual representation of a coverage requirement of one or more portions of the healthcare facility.

14. The computer implemented system of claim 13, wherein the first period of time is 60 days and the second period of time is 30 minutes.

15. The computer implemented system of claim 13, wherein the coverage data comprises one or more of default schedule data, contract staffing requirement data, relative value unit capacity data, service provider types data, cost by provider type data, additional acuity level data, predefined staffing requirements based on the acuity level, contract staffing rules data, and patient treatment related data.

16. The computer implemented system of claim 15, wherein the coverage rules data comprises one or more of a length of a coverage shift, time period limitations on the shift, and predetermined number of shifts per healthcare facility.

17. The computer implemented system of claim 16, wherein the coverage plan matches staff coverage needs in one or more selected time increments for a selected period of time.

18. The computer implemented system of claim 17, wherein the coverage plan further includes one or more coverage recommendations based on coverage needs and costs per shift for the healthcare facility.

19. The computer implemented system of claim 18, further comprising a user interface generator for generating in response to the overage plan one or more user interfaces for displaying the coverage plan.

20. The computer implemented system of claim 19, wherein the user interface generator generates a first user interface having a first window configured for displaying a selected calendar view in a calendar format, wherein each day of the calendar view can display therein selected healthcare related data, including a number of staff scheduled to work each day as well as the expected patient volume for each day.

21. The computer implemented system of claim 19, wherein the first window further comprises an optimization soft button that when actuated optimizes the healthcare related data displayed in the calendar view.

22. The computer implemented system of claim 21, wherein when a selected day in the calendar view is selected by a user, the user interface generator generates a second interface having a second window having a plurality of vertically stacked pane elements for displaying various types of information, wherein the plurality of vertically stacked pane elements includes an uppermost pane element that displays information associated with the coverage plan generated by the coverage determination unit, a second intermediate pane element for displaying selected staff coverage information associated with the coverage plan and RVU information, and a third lowermost pane element for displaying staffing information associated with the coverage plan.

23. The computer implemented system of claim 22, wherein the user interface generator generates a third user interface having a third window for displaying a forecast accuracy dashboard associating an accuracy value associated the patient volume or the staff coverage of the coverage plan for one or more days.

24. A computer implemented method for determining a coverage plan for a healthcare facility, comprising
applying, using an electronic device, a forecasting model to patient volume data received from a data storage unit and then generating in response patient volume forecast data, wherein the forecasting model includes a plurality of hyperparameters and the patient volume data covers a patient volume over a first selected period of time,
tuning, using the electronic device, one or more of the plurality of hyperparameters by applying thereto a tuning technique, wherein the tuning unit generates in response tuned patient volume forecast data,
generating, using the electronic device and with a simulation engine, simulation data in response to the tuned patient volume forecast data and acuity level data, wherein the simulation data includes the patient volume over a second period of time, wherein the second period of time is shorter than the first period of time, and
generating, using the electronic device, a coverage plan in response to and based on the simulation data, coverage data received from a data storage unit, and coverage rules data from a coverage rules unit.

25. The computer implemented method of claim 24, further comprising training the forecasting model with patient volume data as an input and correlating the patient volume data with the healthcare facility.

26. The computer implemented method of claim 25, further comprising simulating, with the simulation engine, the coverage plan based on the tuned patient volume forecast data, the acuity level data, and lay-out data of the healthcare facility.

27. The computer implemented method of claim 26, wherein the simulation data includes a visual representation of a coverage requirement of one or more portions of the healthcare facility.

28. The computer implemented method of claim 27, wherein the coverage data comprises one or more of default schedule data, contract staffing requirement data, relative value unit capacity data, service provider type data, cost by provider type data, additional acuity level data, predefined staffing requirements based on the acuity level, contract staffing rules data, and patient treatment related data.

29. The computer implemented method of claim 28, wherein the coverage rules data comprises one or more of a length of a coverage shift, one or more time period limitations on the shift, and a predetermined number of shifts per healthcare facility.

30. The computer implemented method of claim 29, wherein the coverage plan matches staff coverage needs in one or more selected time increments for a selected period of time with the healthcare facility.

31. The computer implemented method of claim 30, wherein the coverage plan further includes one or more coverage recommendations based on coverage needs and costs per shift for the healthcare facility.

32. The computer implemented method of claim 31, further comprising generating, with the electronic device, a user interface in response to the coverage plan for displaying the coverage plan.

33. A non-transitory, computer readable medium comprising computer program instructions tangibly stored on the computer readable medium, wherein the computer program instructions are executable by at least one computer processor to perform a method, the method comprising:
applying a forecasting model to patient volume data received from a data storage unit and then generating in response patient volume forecast data, wherein the forecasting model includes a plurality of hyperparameters and the patient volume data covers a patient volume over a first selected period of time,
tuning one or more of the plurality of hyperparameters by applying thereto a tuning technique, wherein the tuning unit generates in response tuned patient volume forecast data,
generating with a simulation engine simulation data in response to the tuned patient volume forecast data and acuity level data, wherein the simulation data includes the patient volume over a second period of time, wherein the second period of time is shorter than the first period of time, and
generating a coverage plan in response to and based on the simulation data, coverage data received from a data storage unit, and coverage rules data from a coverage rules unit.

34. The computer readable medium of claim 33, further comprising training the forecasting model with historical patient volume data as an input and correlating the historical patient volume data with the healthcare facility.
